(19) United States
(12) Patent Application Publication
(10) Pub. No.: US 2006/0142831 A1
(43) Pub. Date: Jun. 29, 2006

(54) Limited use ECG electrode set

(75) Inventors: Richard C. Nova, Kirkland, WA (US); Kevin K. Covey, Marysville, WA (US)

Correspondence Address: INGRASSIA FISHER & LORENZ, P.C., 7150 E. CAMELBACK, STE. 325, SCOTTSDALE, AZ, 85251, US

(73) Assignee: Medtronic Emergency Response Systems, Inc.

(21) Appl. No.: 11/024,138

(22) Filed: Dec. 28, 2004

(51) Int. Cl. A61N 1/04

(52) U.S. Cl. 607/142

(57) ABSTRACT

Methods and apparatus are provided for a limited use ECG electrode set. The electrode set includes a plurality of limited use electrodes capable of being affixed to a human patient so as to receive patient information from the patient such as ECG data. Cables are also affixed to each electrode, and each cable is capable of transmitting patient information therethrough. A connector is affixed to each cable, and the connector is likewise capable of transmitting patient information. A sealing wedge may be molded around each cable forming a seal therebetween. The electrode set may be disposed at least partially in a packaging interior region, and the packaging may be hermetically sealed. Further the packaging may be sealed around the sealing wedge in forming the hermetic seal. The limited use electrode set may be opened and deployed from its packaging in situations that call for receiving patient data such as ECG information.

### FIELD OF THE INVENTION

The present invention generally relates to external defibrillators including Automated External Defibrillators (AEDs), and more particularly relates to electrode sets, including electrocardiogram (ECG) electrodes for use with defibrillators. Still more particularly, the present invention relates to single use ECG electrode sets and methods for packaging the same.

### BACKGROUND OF THE INVENTION

Defibrillators are devices for providing life-saving electrical shock therapy to persons experiencing an irregular heart beat, such as ventricular fibrillation. A defibrillator provides an electrical shock to the heart, in order to convert the irregular heart beat to a normal sinus rhythm. Various kinds of defibrillators exist. One type of defibrillator is surgically implanted in patients who are considered likely to need electrical shock therapy, precluding the necessity of constant monitoring by medical personnel. Another commonly used type of defibrillator is the external defibrillator, which sends electrical shock pulses to the patient's heart through external electrodes applied to the patient's chest. External defibrillators may be manually operated, as are typically used in hospitals by medical personnel or may be semi-automatic or fully automatic automated devices, where they can be used in any location where an unanticipated need may occur. An automated external defibrillator is commonly referred to as an AED.

In a typical usage of an external defibrillator, the defibrillator electrodes are attached to the patient prior to delivery of a defibrillation shock. However, often the defibrillator can also be used to monitor the patient's physical condition and related parameters. Patient data can be measured and analyzed, and then a defibrillation decision can be determined based on that analysis. The defibrillator then charges to an appropriate level and applies the shock therapy in a desired format. One or more of these activities can be done by medical/emergency personnel, as in the case of manual defibrillators, or by automated defibrillators. While defibrillators of various designs have been known for several years, there is still room to improve their design.

One example of a need for improvement relates to the defibrillator function of monitoring a patient's physical data. It sometimes happens that a defibrillator is brought to a patient where it is clear that defibrillation therapy may not be required. For example, an ambulance or other emergency responder will be summoned for a patient suffering from chest pains. It later turns out that the patient is not suffering from sudden cardiac arrest or other cardiopulmonary trauma. The patient's heart may be functioning. Another example may be a patient who suffers a minor heart attack but, when EMS arrives, the patient's heart is beating. With respect to these patients, and others, while defibrillation therapy may not be called for, it may still be desired to receive ECG data from the patient. In these kinds of situations, it would thus be desired to have a means of quickly receiving ECG data into a defibrillator. It would further be desired to obtain the patient ECG in a cost-effective method.

The problem relates to the electrodes. Electrodes designed for use with a defibrillator are typically multifunction electrodes in that they are configured both to provide defibrillation therapy and to sense one or more physiologic and/or physical parameters of the patient. This is a typical configuration in an AED type device; it will be understood by those skilled in the art that electrodes may be designed differently for different machines. This kind of electrode can be expensive when used solely for ECG (or other data) purposes. One inherent problem is that a multifunction electrode is designed and manufactured with more functional capability (defibrillation and data transmission) than is being used. Multifunction electrodes are thus typically larger and more expensive than is needed for electrodes handling data transmission functions. It can be wasteful to repeatedly use and dispose of multifunction electrodes when only ECG-type electrodes are needed. It is also advantageous to design the electrodes in a packaged environment for a single use. Reusable, multifunction electrodes must be cleaned, prepared and properly stored after each use.

Hence there exists a need for an improved defibrillator electrode set. It would be desired to provide an improved defibrillator electrode set that is easy to use and that presents an economic advantage over existing methods. Finally, it would be desired to provide a defibrillator set that, by virtue of the foregoing, offers an improved level of response and patient treatment. The present invention addresses one or more of these needs.

### BRIEF SUMMARY OF THE INVENTION

In one embodiment, and by way of example only, there is provided an electrode assembly for quickly providing patient information comprising: a plurality of limited use electrodes capable of being affixed to a human patient and also capable of receiving patient information from the patient; a plurality of cables wherein each cable is affixed to each electrode and wherein each cable is capable of transmitting patient information; a sealing wedge wherein each cable is molded in the sealing wedge; a connector affixed to each cable capable of being connected with a defibrillator and also capable of transmitting patient information to the defibrillator; a packaging defining an interior region and an exterior region; wherein each electrode is disposed within the interior region of the packaging; wherein the packaging is sealed; wherein the connector is in the exterior region of the packaging; and wherein the packaging is sealed around the sealing wedge. Patient information may include information such as electrical, acoustical, chemical, thermal, or optical data resulting in measurements such as ECG, respiration, transthoracic impedance, temperature, perfusion, and blood gas and chemistry related measurements such as oxygen saturation. Gel may be disposed on the electrodes and the packaging sealed so as to protect the gel from degradation. The seal may be a hermetic seal or a seal so as to protect the gel from degradation for the useful lifetime of the electrode assembly. The packaging may be sealed by heat sealing and/or glue.

In a further embodiment, and still by way of example only, there is provided an electrode system for use with a defibrillator comprising: at least one electrode capable of being affixed to a human patient and also capable of receiving ECG information from the patient; a cable connected to the electrode capable of transmitting ECG information; a connector connected to the cable capable of a quick connection with a defibrillator and also capable of transmitting ECG information to the defibrillator; wherein the electrode is a limited use electrode; and wherein the electrode, cable, and connector are capable of being stored together for use with a defibrillator. The electrode system may include any number of electrodes (specifically including sets of two, three, five, and ten) and cables wherein each cable is connected to a single, common connector. The electrode may be a single use or disposable electrode.

Other independent features, characteristics, and advantages of the limited use electrode set will become apparent from the following detailed description, taken in conjunction with the accompanying drawings which illustrate, by way of example, the principles of the invention.

### BRIEF DESCRIPTION OF THE DRAWINGS

FIG. 1 is a schematic diagram of a limited use ECG electrode set according to one embodiment of the present invention.

FIG. 2 is an illustration of a sealing wedge and cables sealed therein according to an embodiment of the present invention.

FIG. 3 is a schematic view of a sealing wedge with cables disposed therein positioned in a packaging opening according to an embodiment of the present invention.

FIG. 4 is an illustration of an external defibrillator system connected to a patient in accordance with an exemplary embodiment of the present invention.

### DETAILED DESCRIPTION

The following detailed description of the invention is merely exemplary in nature and is not intended to limit the invention or the application and uses of the invention. Furthermore, there is no intention to be bound by any expressed or implied theory presented in the preceding background of the invention or the following detailed description of the invention. Reference will now be made in detail to exemplary embodiments of the invention, examples of which are illustrated in the accompanying drawings. Wherever possible, the same reference numbers will be used throughout the drawings to refer to the same or like parts.

It has now been discovered that a set of ECG electrodes that are designed to have a single use or a limited use can be utilized with current defibrillator devices. The electrodes are preferably relatively inexpensive as compared to therapeutic or multifunction ECG/therapeutic electrodes. The electrodes are advantageously used when, for example, it is desired to monitor a patient's heart function without the need to provide therapeutic defibrillation. The single use ECG electrodes provide the needed patient information with a cost savings over ECG/therapeutic electrodes. The design and packaging of the electrode set also allows for their quick and efficient use.

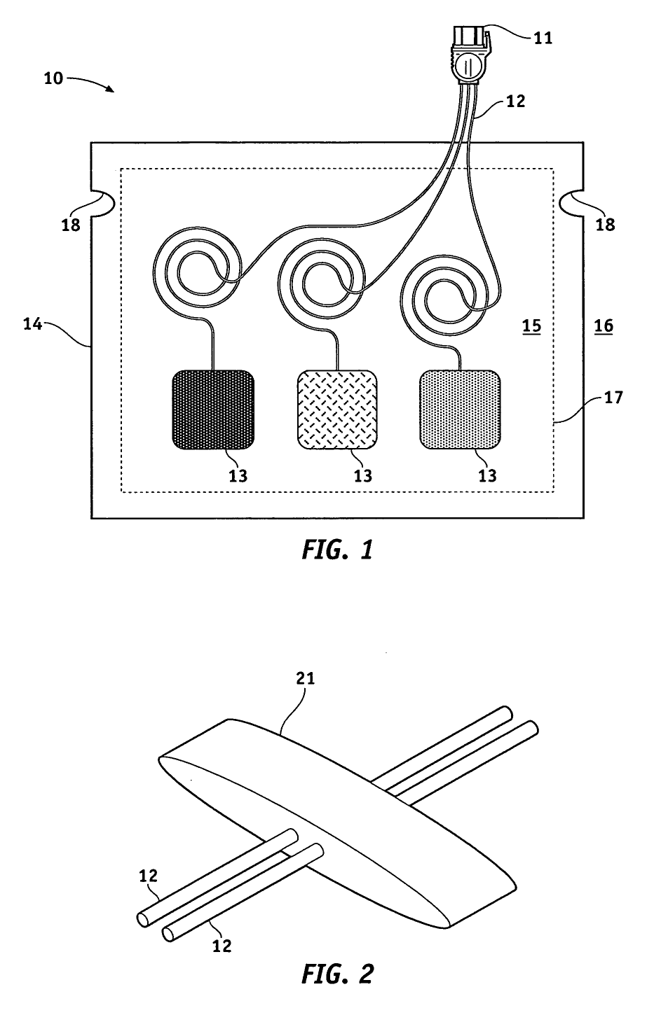

In an embodiment of the invention, there is provided a limited-use ECG electrode pad, cable, and connector set. Referring now to FIG. 1 there is shown a preferred embodiment of the limited use ECG electrode set. The set 10 comprises a connector 11, cable 12, and electrode 13. Preferably, the electrode 13, cable 12, and connector 11 are permanently or semi-permanently attached to one another. Cable 12 typically has two ends. In the preferred embodiment, one cable 12 is matched with one electrode 13. As is known in the art, cable 12 includes a transmission material in its interior (not shown) surrounded by an insulation material.

Connector 11 is configured so that it may be plugged into a port or receptacle of a defibrillator. As is known in the art, connector 11 thus may have a shape that can be received by the defibrillator. Further, connector 11 may be designed so that it plugs into a multifunction port and/or data port of the defibrillator. Connector 11 may be fabricated of materials suitable for collection and transmission of patient data.

In the typical attachment, electrode 13 is attached to cable 12 at one end of cable 12, and connector 11 is attached to cable 12 at the opposite end of cable 12. The form of attachment may be any kind known in the medical electrode art including, but not limited to, soldering, crimping, adhesive, and mechanical (screw) attachment. Also, as is known in the art, the attachment is such that patient information, particularly ECG information, may be transmitted through electrode 13, through cable 12, through connector 11, and into a defibrillator (or ECG monitor).

In this embodiment, the electrodes are connected to the cable and/or a connector set. The set is designed for single use. Additionally the set is connected to a defibrillator, through the connector, when intended for use.

Also shown in FIG. 1 is packaging 14. In a preferred embodiment packaging 14 comprises a flexible material. Packaging 14 defines an interior region 15 and exterior region 16. Packaging 14 may be comprised of two sheets which are joined together to define packaging 14. Packaging 14 may be sealed, and when it is the interior region 15 is isolated from the environment in the exterior region 16. A seal 17 is illustrated in FIG. 1.

The degree of sealing that is provided by packaging 14 is preferably a sealing sufficient to protect materials packaged with electrodes 13 in the packaging interior 15. As is understood in the art, electrodes 13 may be stored with gel-like materials on their surfaces. This gel-like material serves the dual purpose of protecting the metals of the electrode against potential degradation. Further, when placed on a patient, the gel materials provide a helpful interface between the electrodes and the patient body. Thus, it is generally important to preserve and maintain the gel against evaporation or degradation. Often the gels are water-based. Exposure to an environment of heat and/or low humidity may quicken the evaporation of moisture in the gel. Hence, the degree of sealing is preferably sufficient so as to prevent gel degradation below performance levels during the expected lifetime of the electrode 13.

When it is desired to use packaging 14 with electrode set 10, the materials of the electrode set 10 may be disposed completely, or partially, within the interior region of packaging 14. In a preferred embodiment shown in FIG. 1, electrode set 10 is disposed partially within the interior region 15. It is noted that cables 12 cross from the exterior 16 to the interior 15 of the packaging 14. In a preferred embodiment, cables 12 are disposed in a structure that allows good sealing where cables 12 cross the seal 17.

Referring now to FIG. 2 there is shown a set of electrode cables 12 molded in a structure identified herein as a sealing wedge 21. The sealing wedge 21 is preferably a plastic material. Molding cables 12 within sealing wedge 21 allows for a sealing at the point where cables 12 actually pass through the sealing wedge structure.

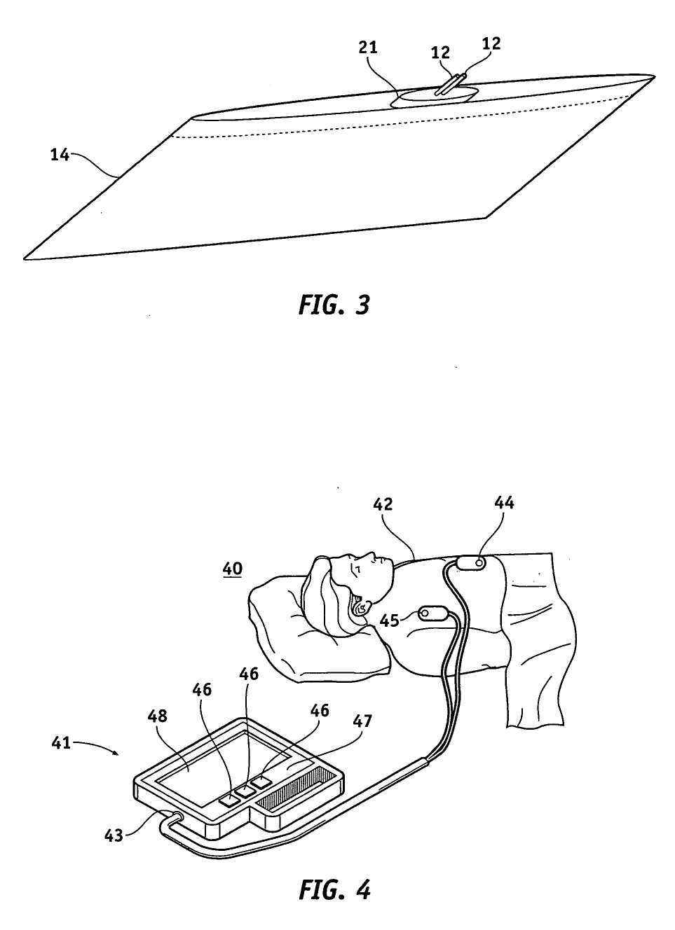

Referring now to FIG. 3 there is shown a sealing wedge 21 positioned for sealing into packaging 14. When sealing wedge 21 is placed in a packaging seam, the seam may then be heat sealed. Both sides of the packaging are thereupon bonded to the sealing wedge 21. This results in a hermetically sealed pouch. Cables 12 protrude from the sealing wedge, and through the pouch; however, the seal is maintained. As shown in FIG. 2 sealing wedge 21 is preferably elliptical in shape with a smooth upper surface and lower surface. This shape, and the smoothness of the surfaces, allows for a good fit and sealing in packaging 14. Preferably cable insulation and sealing wedge 21 are selected of materials that are compatible so as to provide good sealing when cables are molded into the sealing wedge. Additionally, sealing wedge 21 and packaging material are also selected of compatible materials so that heat sealing the packaging allows a sealing closure with sealing wedge 21.

Referring again to FIG. 1 it is noted that cables 12 are drawn with the majority of their length positioned in the interior region 15 of packaging 14. Optionally, cables 12 can be disposed with any length of cable in the interior region 15 or exterior region 16 of the packaging 14. It is generally preferred, however, to position the majority of the cable length in the interior 15. This provides the benefit of avoiding lengths of cable 12 that can potentially become entangled or damaged in the exterior 16 of the packaging 14.

It is further noted that positioning the majority of the cable length within the packaging interior 15 is advantageous when sealing the cables 12 with a sealing wedge 21. Referring still to FIG. 1 there is shown a set of cables 12 with only a small length of cable 12 protruding through the packaging 14. Though the sealing wedge 21 is not illustrated in this figure, when sealing wedge 21 is present, it would be positioned along the seal 17 where cables 12 pass from interior region 15 to exterior region 16 of the packaging 14. Thus, as shown in this figure, just as there is a short length of cable 12 between packaging 14 and connector 11, similarly there would be a short length of cable between sealing wedge 21 and connector 11. The distance of cable 12 between sealing wedge 21 and connector 11 may be short such as less than five inches or less than half the overall length of cable 12. This is advantageous because, once an electrode set 10 is removed from packaging 14, the sealing wedge 21 will remain in place holding cables 12. The majority of the length of cable 12 will be free to swing about for positioning on the patient while tethered to its molded position in sealing wedge 21. This gives maximum freedom to position electrodes 13 as needed.

In an optional embodiment, the portion of cable 12 that is disposed within the interior region 15 of packaging 14 may be wound, wrapped, or tied in order to prevent the cable 12 therein from becoming tangled. This also provides the advantage that, upon breaking the packaging 14 and using the electrode set 10, the cable 12 may be quickly and efficiently unwound.

Several procedures can be used to seal packaging 14. In one embodiment, the packaging pouch is sealed under conditions of ambient air and ambient pressure. The seal is obtained by melting the heat sealable pouch material. In this sealing embodiment, it may be advantageous to capture a bubble of air in the packaging interior during the sealing procedure. The air bubble may serve to inflate the packaging thereby protecting the interior contents from crushing or other ill-effects that occur during storage and shipping. Optionally, or additionally, the sealing bond may be achieved through the use of adhesives. In another embodiment, the packaging may be vacuum packaged. In still another embodiment, the packaging material may be inflated with an inert gas during packaging thereby sealing the inert gas in the interior region of the packaging.

As is known in the art, the limited use electrode set 10 can be provided with various numbers of electrodes 13 and cables 12. Also, as is known, different combinations and numbers of electrodes 13 can be used to receive different kinds of patient data and/or data of a higher or lower quality. Different numbers of electrodes can generate different sets of “leads” which may be described as looks or views of some area of the patient, such as the heart muscle. A set of three electrodes 13, as illustrated in FIG. 1, is one electrode combination that can be used to receive one kind of patient data in a known format. Also, a ten electrode 13/ten cable 12 set is another combination of electrodes and cables that can be used to receive patient data in a different format. It will thus be understood by those skilled in the art that the limited use electrode set is not limited in the number or kinds of cables 12 and electrodes 13 that may be used therein. In a preferred embodiment multiple sets of electrodes and cables are connected to a single, common connector.

Referring again to FIG. 1 it is also noted that tear marks 18 are included in packaging 14. As shown tear marks 18 can include partial perforations of the material of the packaging. Alternatively, other means that help guide a tear may be employed. Tear marks 18 thus identify a portion of packaging 14 that allows a user to quickly open a sealed packaging 14.

The electrode set 10 is a set intended for limited use or single use (disposable). As is understood in the art, the electrodes thus are not designed or intended for multiple uses. As compared to reusable electrodes, electrodes 13 of the limited use electrode set 10 are comparatively smaller in area. Additionally electrodes 13 of the limited use electrode set 10 comprise comparatively thinner layers of transmissive material thereon as compared to multifunction or reusable electrodes. A limited use electrode set 10 is also characterized in that electrodes 13 are not designed to provide defibrillation therapy, and may be designed to inhibit delivery of defibrillation therapy. Additionally cables 12 may be designed so as to inhibit delivery of defibrillation therapy from a defibrillator therethrough. In some embodiments, connector 11 may also be designed to inhibit passage of defibrillation therapy therethrough. Further, an electrode set 10 may include an electronic valve or inhibitor so as to avoid defibrillation therapy passing through the set.

Having described the limited use electrode set from a structural standpoint, a method of using the same will now be described.

In one embodiment, a packaged electrode set 10 is brought to the scene of an emergency. A companion defibrillator is also present. It is preferred to store a packaged electrode set 10 with a defibrillator or device with which it is to be used. In one embodiment, the defibrillator may include a compartment or other storage device so as to simultaneously transport a packaged electrode set 10 with the defibrillator.

At the emergency scene, a user determines that ECG information (or other patient data) should be obtained. The user thereupon opens packaging 14. Connector 11 is plugged into the defibrillator, and electrodes 13 are positioned on the patient. Patient information is thereupon received by electrodes 13, transmitted through cables 12, through connector 11, and into the defibrillator. Defibrillator can then receive, process, and output patient data through known methods.

In a further embodiment, different types of packaged electrode sets 10 are brought to the scene of an emergency along with a defibrillator. The user determines what kind and quality of patient data to measure. For example, the user may decide between using a three-electrode set versus a ten-electrode set. Upon making the decision, the user selects the appropriate packaged electrode set, opens it, and deploys the set as described above. In a further use of the electrode sets, it is possible to switch between different types of electrode sets 10. Thus, in the example of selecting between three-electrode and ten-electrode sets, it is possible to begin receiving patient data through a three-electrode set and then switch to a ten-electrode set. It is also possible to do the reverse.

Referring now to FIG. 4 there is shown a typical defibrillator system 40 that may be used with embodiments of the present invention. It will be understood that this defibrillator system is merely exemplary of a defibrillator that may be used with a limited use electrode set. The defibrillator system 40, includes, but is not limited to, an external defibrillator 41 having a connection port 43 that is configured to receive one or more electrodes (44, 45). (A defibrillator may also have more than one connection port). FIG. 4 illustrates a defibrillator having two electrodes, but it will be understood from the previous discussion that a different number is possible. The external defibrillator 41 can be any number of external defibrillators in accordance with the present invention. For example, the external defibrillator 41 can be an Automated External Defibrillatoror a manually operated external defibrillator. U.S. Pat. No. 4,610,254 to Morgan and U.S. Pat. No. 6,334,070 to Nova provide illustrative examples of defibrillators, and these two patents are hereby incorporated in their entirety by reference.

The external defibrillator 41 may include a user interface 47. The interface 47 may include an output device such as a display 48 that is configured to visually present information which may include various measured or calculated parameters of patient 42 and/or other information to the operator (not shown) of the external defibrillator 41. Display 48 may provide information in textual, numeric, graphical, and/or symbolic format. Information may also be output from the defibrillator through other means such as but not limited to audible signals and/or voice prompts through a speaker or other audio generation device.

The user interface 47 can also include one or more input devices 46 that are configured to receive commands or information from the operator. Input devices may include, but are not limited to, devices such as keys, buttons, switches, touch screens, keyboards, and keypads.

The one or more electrodes (44, 45) are also configured to sense one or more physiologic and/or physical parameters of the patient 42 that are received by the external defibrillator 41 at the connection port 43. The signals provided by the one more electrodes (44, 45) may be evaluated by the external defibrillator 41 and displayed through output device display 48.

A variety of physiological data and signals of the patient 42 can be sensed by the defibrillator. For example, conventional phonocardiogram (PCG) transducers can be used to convert acoustical energy of the patient's heart to electrical energy for production of a PCG waveform. Additionally, electrical activity of the patient's heart can be converted for production of an electrocardiogram (ECG) waveform. Transthoracic impedance and other physiology signals of the patient may also be detected. This data represented by this information can be collected and processed in the controller of the defibrillator.

In view of the foregoing, it should be appreciated that embodiments of a limited use ECG electrode set are described herein. While a finite number of exemplary embodiments have been presented in the foregoing detailed description of the invention, it should be appreciated that a vast number of variations exist. It should also be appreciated that the exemplary embodiments are only examples, and are not intended to limit the scope, applicability, or configuration of the invention in any way. Rather, the foregoing detailed description will provide those skilled in the art with a convenient road map for implementing exemplary embodiments of the invention. It thus should be understood that various changes may be made in the function and arrangement of elements described in an exemplary embodiment without departing from the scope of the invention as set forth in the appended claims.

What is claimed is:

1. An electrode system for use with a defibrillator comprising:
at least one electrode capable of being affixed to a human patient and also capable of receiving ECG information from the patient;
a cable connected to the electrode capable of transmitting ECG information;
a connector connected to the cable capable of a connection with a defibrillator and also capable of transmitting ECG information to the defibrillator;
wherein the electrode is not intended for delivery of defibrillation therapy; and
wherein the electrode is a limited use electrode; and
wherein the electrode, cable, and connector are capable of being stored together for use with a defibrillator.

2. The electrode system according to claim 1 further comprising a plurality of electrodes and cables wherein each cable is connected to a single, common connector.

3. The electrode system according to claim 1 further comprising three electrodes wherein each electrode is attached to a separate cable.

4. The electrode system according to claim 1 further comprising ten electrodes wherein each electrode is attached to a separate cable.

5. The electrode system according to claim 1 further comprising a pair of electrodes, each electrode affixed to a separate cable, and each cable affixed to a single, common connector.

6. The electrode system according to claim 1 wherein the electrode is a single use electrode.

7. The electrode system according to claim 1 wherein the electrode is a disposable electrode.

8. An electrode assembly for quickly providing patient information comprising:
a plurality of limited use electrodes capable of being affixed to a human patient and also capable of receiving patient information from the patient;
a plurality of cables wherein each cable is affixed to each electrode and wherein each cable is capable of transmitting patient information;
a sealing wedge wherein each cable is molded in the sealing wedge;
a common connector affixed to each cable capable of being connected with a defibrillator and also capable of transmitting patient information to the defibrillator;
a packaging defining an interior region and an exterior region;
wherein each electrode is disposed within the interior region of the packaging;
wherein the packaging is sealed;
wherein the connector is in the exterior region of the packaging; and
wherein the packaging is sealed around the sealing wedge.

9. The electrode assembly according to claim 8 wherein patient information comprises ECG information.

10. The electrode assembly according to claim 8 wherein patient information comprises electrical information.

11. The electrode assembly according to claim 8 wherein patient information comprises transthoracic impedance information.

12. The electrode assembly according to claim 8 wherein patient information comprises acoustical information.

13. The electrode assembly according to claim 8 further comprising a gel disposed on the electrodes and wherein the packaging is sealed so as to protect the gel from degradation.

14. The electrode assembly according to claim 13 wherein the packaging is sealed so as to protect the gel from degradation for the useful lifetime of the electrode assembly.

15. The electrode assembly according to claim 8 wherein the packaging is sealed so as to isolate the interior region of the packaging from the exterior region.

16. The electrode assembly according to claim 8 wherein the packaging is hermetically sealed.

17. The electrode assembly according to claim 8 wherein the packaging is sealed by a heat sealing.

18. The electrode assembly according to claim 8 wherein the packaging comprises a material that is tearable by a human user.

19. The electrode assembly according to claim 8 further comprising a bubble of air disposed in the interior region of the packaging.

20. The electrode assembly according to claim 8 wherein the packaging is sealed using glue.

21. The electrode assembly according to claim 8 wherein inert gas is disposed within the interior region of packaging.

22. The electrode assembly according to claim 8 wherein the electrode assembly is a limited use assembly.

23. The electrode assembly according to claim 8 wherein the electrode assembly is a single use assembly.

24. The electrode assembly according to claim 8 wherein the electrode assembly is disposable.

25. The electrode assembly according to claim 8 wherein the sealing wedge is elliptical in shape.

26. An electrode assembly for quickly providing patient information comprising:
a plurality of limited use electrodes capable of being affixed to a human patient and also capable of receiving patient information from the patient;
a plurality of cables wherein each cable is affixed to each electrode and wherein each cable is capable of transmitting patient information;
a common connector affixed to each cable capable of being connected with a defibrillator and also capable of transmitting patient information to the defibrillator;
a packaging defining an interior region and an exterior region;
wherein each electrode, connector, and cable is disposed within the interior region of the packaging; and
wherein the packaging is sealed.

27. The electrode assembly according to claim 26 wherein patient information comprises ECG information.

28. The electrode assembly according to claim 26 wherein patient information comprises electrical information.

29. The electrode assembly according to claim 26 wherein patient information comprises transthoracic impedance information.

30. The electrode assembly according to claim 26 wherein patient information comprises acoustical information.

31. The electrode assembly according to claim 26 further comprising a gel disposed on the electrodes and wherein the packaging is sealed so as to protect the gel from degradation.

32. The electrode assembly according to claim 31 wherein the packaging is sealed so as to protect the gel from degradation for the useful lifetime of the electrode assembly.

33. The electrode assembly according to claim 26 wherein the packaging is sealed so as to isolate the interior region of the packaging from the exterior region.

34. The electrode assembly according to claim 26 wherein the packaging is hermetically sealed.

35. The electrode assembly according to claim 26 wherein the packaging is sealed by a heat sealing.

36. The electrode assembly according to claim 26 wherein the packaging comprises a material that is tearable by a human user.

37. The electrode assembly according to claim 26 further comprising a bubble of air disposed in the interior region of the packaging.

38. The electrode assembly according to claim 26 wherein the packaging is sealed using glue.

39. The electrode assembly according to claim 26 wherein inert gas is disposed within the interior region of packaging.

40. The electrode assembly according to claim 26 wherein the electrode assembly is a limited use assembly.

41. The electrode assembly according to claim 26 wherein the electrode assembly is a single use assembly.

42. The electrode assembly according to claim 26 wherein the electrode assembly is disposable.

43. A method of packaging a single use electrode for use in transmitting patient information to a defibrillator, the method comprising the steps of:
molding a sealing wedge around a cable with two ends;
attaching an electrode at one end of the cable wherein the electrode is not intended for delivery of defibrillation therapy;
attaching a connector to the opposite end of the cable;
disposing the electrode in the interior region of a packaging; and
sealing the packaging so as to preserve the electrode for emergency use.

44. The method according to claim 43 wherein patient information comprises ECG information.

45. The method according to claim 43 wherein patient information comprises electrical information.

46. The method according to claim 43 wherein patient information comprises acoustic information.

47. The method according to claim 43 further comprising positioning the sealing wedge around the cable so as to allow a short cable distance between the sealing wedge and the connector.

48. The method according to claim 43 further comprising positioning the sealing wedge around the cable so as to allow a cable distance between the sealing wedge and the connector that is less than the overall length of the cable.

49. The method according to claim 43 further comprising positioning the sealing wedge around the cable so that a length of less than five inches of cable exists as measured between the point where the cable enters the sealing wedge and the point where the cable enters the connector.

50. The method according to claim 43 wherein the step of sealing the packaging further comprises trapping an air bubble in the interior region of the packaging.

51. The method according to claim 43 wherein the step of sealing the packaging further comprises hermetically sealing the packaging.

52. The method according to claim 43 further comprising tearing open the packaging.

53. The method according to claim 43 further comprising inserting the connector into a defibrillator connection port and placing the electrode on a patient so as to receive ECG information.

54. The method according to claim 43 wherein the step of molding a sealing wedge further comprises molding an elliptical shaped sealing wedge.

55. A method of packaging a single use electrode for use in transmitting patient information to a defibrillator, the method comprising the steps of:
attaching an electrode at one end of a cable with two ends, wherein the electrode is not intended for delivery of defibrillation therapy;
attaching a connector to the opposite end of the cable;
disposing the electrode, cable, and connector in the interior region of a packaging; and
sealing the packaging so as to preserve the electrode for emergency use.

56. The method according to claim 55 wherein patient information comprises ECG information.

57. The method according to claim 55 wherein patient information comprises electrical information.

58. The method according to claim 55 wherein patient information comprises acoustic information.

59. The method according to claim 55 wherein the step of sealing the packaging further comprises trapping an air bubble in the interior region of the packaging.

60. The method according to claim 55 wherein the step of sealing the packaging further comprises hermetically sealing the packaging.

61. The method according to claim 55 further comprising tearing open the packaging.

62. The method according to claim 55 further comprising inserting the connector into a defibrillator connection port and placing the electrode on a patient so as to receive ECG information.
